(19) United States
(12) Patent Application Publication
(10) Pub. No.: US 2023/0293964 A1
(43) Pub. Date: Sep. 21, 2023

(54) DYNAMIC FEEDBACK SCHEDULES

(71) Applicant: Weav Music, Inc, Brooklyn, NY (US)

(72) Inventors: Lars Eilstrup Rasmussen, Brooklyn, NY (US); Mansimran Singh, Brooklyn, NY (US); Keit Kollo, Brooklyn, NY (US); Konstantinos Tsitsas, Brooklyn, NY (US)

(21) Appl. No.: 18/000,465

(22) PCT Filed: May 27, 2021
(86) PCT No.: PCT/GB2021/051305

§ 371 Date: Dec. 1, 2022

(30) Foreign Application Priority Data
Jun. 1, 2020 (GB) 2008210.3

(51) Int. Cl. A63B 71/06; A63B 24/00

(52) U.S. Cl. CPC A63B 71/0622; A63B 24/0062; A63B 24/0075; A63B 2024/0068; A63B 2071/063

(57) ABSTRACT

A computer-implemented method for controlling playback of a schedule of feedback elements to be presented at an output of an electronic device, the feedback elements each associated with one or more predefined metrics, includes the steps of, during playback of the schedule: receiving sensor data from one or more sensors associated with the electronic device; upon determining that a first trigger condition is satisfied, determining whether the sensor data satisfies a metric threshold condition, in response to the metric threshold condition being satisfied, modifying the schedule of feedback elements for presenting at the output. An associated computer readable medium and electronic device are also described.

### CROSS REFERENCE

This application is a U.S. National Phase Application of International Application No. PCT/GB2021/051305 filed on May 27, 2021, which in turn claims priority to GB Application No. 2008210.3 filed on Jun. 1, 2020, the disclosures of which are incorporated herein by reference in their entireties.

### TECHNICAL FIELD

The present disclosure relates generally to a method and electrical device for playback of a schedule of feedback elements to a user.

### BACKGROUND

With the rise of electronic devices that can monitor a user's activity, so has there been a rise in applications which seek to provide a user with feedback on their performance of various physical activities.

Known systems include GPS and other tracking devices that will present a user with an indication of their pace while running, or their cadence when riding a bike, for example. A recent development in the field of fitness is the ability to provide predetermined workouts, that are to be performed by a user. These predetermined workouts often take the form of an audio file containing a mixture of music and spoken feedback elements—such as instructions to begin a specific workout interval, and to indicate that a workout interval is complete.

An improved method is required for providing dynamically updated and generated feedback.

### SUMMARY

According to an aspect, a computer-implemented method is provided for controlling playback of a schedule of feedback elements to be presented at an output of an electronic device, the feedback elements each associated with one or more predefined metrics, the method comprising, during playback of the schedule: receiving sensor data from one or more sensors associated with the electronic device; upon determining that a first trigger condition is satisfied, determining whether the sensor data satisfies a metric threshold condition, in response to the metric threshold condition being satisfied, modifying the schedule of feedback elements for presenting at the output.

Optionally the schedule comprises a first feedback element associated with a first metric, and modifying the schedule comprises providing a second feedback element associated with the first metric, and replacing the first predetermined feedback element with the second feedback element. The first feedback element may be associated with a first range of first metric data and the second feedback element is associated with a second range of first metric data, and selecting the second feedback element is done when the satisfied metric threshold condition is located in the second range.

Optionally, modifying the schedule comprises adding a third feedback element to the schedule for presenting at the output.

Optionally, modifying the schedule comprises modifying a time at which a scheduled feedback element is to be presented at the output.

Optionally, the schedule comprises a schedule of events, each having an associated trigger condition and one or more associated feedback elements, and modifying the schedule comprises, when a first trigger condition of a first event is satisfied, removing the feedback element of a second event from the schedule of feedback elements for presenting at the output.

Optionally, wherein the schedule comprises a schedule of events, each comprising a trigger condition and associated with one or more of the predefined metrics, wherein modifying the schedule comprises, where a first trigger condition of a first event is satisfied, creating a second event comprising a second trigger condition and associated with one or more of the predefined metrics, and adding the second event to the schedule of events.

Optionally, the schedule of feedback elements comprises at least one predetermined feedback element, and modifying the schedule comprises: generating a context-dependent feedback element based on the sensor data, and adding the generated context-dependent feedback element to the schedule for presenting at the output. Generating the context-dependent feedback element may comprise combining a plurality of media samples.

Optionally, the determining whether the metric threshold condition is satisfied comprises comparing the sensor data with stored data associated with one or more of the predefined metrics.

Optionally, the feedback elements comprise audio components.

Optionally, the feedback elements comprise speech components.

Optionally, the first schedule comprises a plurality of intervals each having a length, and a first and second interval boundary, and wherein the first trigger condition is satisfied when during playback of the schedule an interval boundary is crossed.

Optionally, the length of an interval is defined as one of: a function of time or a function of distance.

Optionally, the sensor data relates to one or more of: biometric data gathered by a biometric sensor, location data gathered by a location sensing device, movement data gathered by an inertial measurement unit.

Optionally, the predefined metrics comprise one or user parameters, the user parameters being one or more of: pace; speed; heart rate; cadence; stroke rate; interval completion success, or volume of oxygen consumption (VO2).

According to a further aspect, a computer readable medium is provided comprising computer readable instructions configured, in use, to enable a processor to perform the method steps described herein.

According to a further aspect, an electronic device is provided, arranged to playback a schedule of feedback elements, the electronic device comprising a processor arranged to perform the method steps described herein.

Optionally, the electronic device comprises an audio output, and the feedback elements comprise audio data, and playback of the schedule of feedback elements comprises presenting the feedback elements at the audio output.

### BRIEF DESCRIPTION OF THE DRAWINGS

Examples of the present disclosure will now be explained with reference to the accompanying drawings in which:

FIG. 1 shows a block diagram of a system for implementing the methods described herein;

FIG. 2 is a flowchart for dynamic processing of a schedule based on received sensor data;

FIG. 3 is a flowchart relating to the methods disclosed herein;

FIG. 4 is a flowchart showing an example of processing schedule events;

FIG. 5 is a flow diagram showing operational steps in the context of audio events;

FIG. 6 is a flowchart illustrating composition of audio events; and

FIG. 7 shows an exemplary illustration of the modification of scheduled feedback elements according to the methods described herein.

Throughout the description and the drawings, like reference numerals refer to like parts.

### DESCRIPTION OF EXAMPLE EMBODIMENTS

### Overview

The embodiments of the invention described herein relate to presenting dynamically updated feedback based on sensor data received at an electronic device. A schedule of events is executed, the events being associated with one or more trigger conditions, and associated metrics calculated based on the received sensor data. When, in a schedule of events that is currently being executed, event trigger conditions are met, and metric conditions are also satisfied, feedback elements may be passed to a feedback output generator, and the schedule itself may be dynamically updated. The triggering of event trigger conditions gives rise to dynamically updating one or more of the schedule of events, the trigger conditions associated with one or more subsequent events, the feedback elements associated with a one or more events, or a schedule of feedback elements queued to be played out at an output of the electronic device. In this way, based on the sensor data received, an improved feedback output is provided.

### Example Embodiments

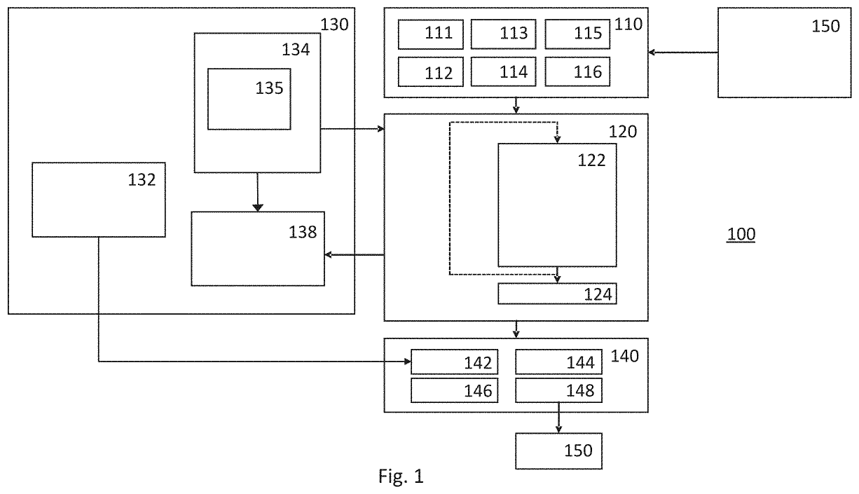

FIG. 1 depicts a user electronic device 100 that can be used for playback of feedback schedules, specifically schedules based on user performance. The user device 100 may be a smartphone, a portable media player or any other electronic device capable of playback of a schedule of feedback elements. The user device 100 comprises an input module 110 comprising one or more input receiving devices. The inputs may include one or more of Clock 111, an altimeter 112, a GPS system 113, accelerometer 114, a gyroscope 115, a music module 116. The accelerator 114 and gyroscope 115 may form part of an inertial measurement unit (IMU). The input module 110 may further comprise, or be associated with other sensors such as a heart monitor, a barometer, a bicycle power meter or similar. The input module 110 is arranged to receive input signals in either a wired or wireless manner, as required. The electronic device 100 further comprises a schedule engine 120, arranged to process feedback schedules, or programmes. The schedule engine 120 may reside on a processor arranged to execute instructions of a computer program to perform the methods described herein. The user device 100 also comprises storage 130 for storing a plurality of schedules, and associated media files which may be used to provide feedback to the user. The storage 130 comprises a feedback element library 132, one or more schedules 134, and may further comprise a user data repository 138. The feedback element library 132 may comprise one or more audio or video files, media files, or data files, which may relate to haptic feedback patterns. The user device 100 further comprises an output unit 140 for providing feedback output. The feedback output 140 may comprise one or more of an audio generator 142, a haptic feedback generator 144 arranged to provide feedback to a user through vibration of either the electronic device itself or an associated haptic feedback device, a feedback transmitter module 146, and a display 148. The audio generator 142 may provide an audio output to a speaker or a headphone jack or combined audio/power connector.

A user of user device 100 may wish to use the device to playback a schedule, for example a workout schedule or programme of instructions to be carried out. A user may wish to be provided with feedback on their performance which allows them to not only monitor their basic performance, but also performance based on a number of predefined metrics which may be obtained and calculated while the schedule is being executed, allowing, for example for the provision of bespoke or tailored feedback. The dynamic schedule presented herein allows for feedback to be presented not only at predetermined points in the schedule, but also to be modified and updated during the playback of the schedule, based on data received from a plurality of sensors associated with the user device 100.

The sensor data that is received and processed by the device may relate to user performance data, such that, for example, location data received by the GPS receiver system 113 can be converted into speed, distance or pace (per unit distance) data. The sensor data received by the accelerometer and the gyroscope may be processed and converted into running cadence (steps per minute), stroke rate (in a swimming context), or pedalling cadence (in a cycling context). Other sensor data may be received from sensors external to the user device—for example present weather conditions, received at the user device from a wired or a wireless network connection, such as Wi-fi or Bluetooth®.

The user device 100 comprises one or more schedules 134 or programmes which are to be played back by the user. A schedule comprises a number of events 135, and score rules 136.

Each schedule comprises, or is associated with, feedback elements or events which are to be provided to the user. The feedback elements may be alerts provided to the user audibly, through the audio output of the electronic device, displayed visually on the display of the electronic device, or provided to the user through haptic feedback.

The schedules provided in the device will now be described. A schedule, program or list, comprises a series of intervals which are to be processed sequentially. Each interval has a defined length—which may be defined in terms of a duration, in units of time, or as a distance, in units of length. For example an interval may be defined simply as a “warm up” interval, with a duration of 5 minutes, or may be defined as being completed when a particular distance is covered by the user. Intervals may be grouped into interval groups. For example, a schedule may comprise four active intervals of 5 minutes, with a rest interval between each active interval. For the purposes of interval processing, as described below, the processing of events associated with “active” and “rest” intervals may be treated the same, or differently. It is possible to have nested interval groups. A nested interval group in a schedule might include 3 repetitions of the interval group described above, with a different longer rest interval between each repetition. Global schedule rules may apply to intervals individually, or to interval groups. The commencement or playback of a particular interval may be contingent on the completion of a previous interval.

Each interval comes with an associated set of events. At the most basic level the interval has a beginning and an end, with each of which an event may be associated. These events are considered as “fixed” events, and are triggered by a specific point in the interval being reached. An interval may comprise “floating” events, which may be triggered at any point in an interval when a trigger condition is met. The events that are associated with an interval may be triggered by real-time input from sensor data received from the user device.

Events may be feedback or non-feedback generating events. Feedback generating events give rise to feedback elements being provided to the user during playback of a schedule, while non feedback-generating events may relate to the storage of particular sensor data or calculated metric data in the storage 130. It is noted that the term “playback” means execution, implementation or sequentially running through the schedule items, and need not be restricted to the associated audio/video connotations of the term playback. Event-related feedback may be audio instructions, or voice clips. These can be pre-recorded or composed in real-time based on sensor input data. In the example of a schedule intended to be used as a running workout, sensor data relating to actual location (from the GPS sensor, for example) may be used to compose an audio clip event containing a user's present running pace. Further examples of events include device vibration using the haptic feedback generator 144, display of information on the display 148. Events can also create further events to be either added to the present interval, or to intervals which appear later in the schedule, as will be described further below. These events have associated rules which when triggered, elicit the creation and scheduling of an event later in the schedule.

Each event is associated with one or more milestones, otherwise referred to herein as metric threshold conditions or metric trigger conditions. These milestones are pre-defined heuristics which are computed based on received sensor data and knowledge about the current schedule condition or the environment. A milestone may be the current position of the schedule in time, based on sensor data received by the GPS receiver 113. Another example of a milestone is the current location of the user. For example an event may be triggered at a specific location, or in relation to a “geofence” which is crossed by the user moving through the environment. A further example of a milestone is current altitude, based on data received from the altimeter. An event may be associated with a particular altitude value and event feedback is triggered when that altitude is reached by the user, or when a predetermined altitude is approaching, either based on a calculated rate of change of altitude, or where a pre-programmed route is used, and the device is approaching a portion of the route including a hill climb. Alternatively an event might be triggered by a rate of change of altitude or pressure, based on processing of sensor data from the altimeter 114 and a barometer associated with the device.

A further example of a milestone is time passed in relation to another event. For example time since start, or time since last instruction, or time left in the current interval. Events can be dependent on a previous event already having been delivered to the user. Equally a milestone can be distance covered in relation to another event—for example 0.5 km since the start of the interval or since a previous linked event was delivered, or where there is 400 m left to the end of an interval that is based on a distance.

Milestones can be any function (average, current, minimum, maximum, total, within range, out of range) of the sensor data received which can be computed as speed, distance, duration, cadence, heart rate, stroke rate. For example an interval may be defined as requiring that an activity is performed within a heart rate range, with lower and upper limits of 140-160, and event feedback is triggered when the received sensor data goes out of this range, or when the received data remains within the range for a predetermined amount of time. Milestones may therefore be nested functions of other milestones—for example maintain the heart rate range above for a specific duration, or maintain a specific pace for a specific time.

Other knowledge about the present conditions of the device, based on the sensor data received, may be used as a milestone or metric trigger condition. This may relate to current music being played on the electronic device, present environmental conditions (temperature, or data received from a weather service), or other upcoming milestones. The input services module 110 is arranged to process the incoming data and provide this data to the feedback engine to determine whether threshold conditions are met. For example a weather alert may be received from an external source—if it is determined to be relevant to the present schedule being executed since the alert will apply during the schedule playback, then feedback may be presented to a user. For example, if rain will begin in 20 minutes' time, and an estimated time for completion of the schedule is 30 minutes, an event trigger condition might be that the estimated completion time is greater than time until weather notice starts, and appropriate feedback can be scheduled by the feedback schedule engine 120.

A further example of a milestone can be a calculated rate of perceived exertion, or RPE. In the context of a running workout, this may be calculated as a function of pace (speed/time) combined with other data such as running cadence and heart rate data. In the context of a cycling workout this may be related to power output, measured by a power meter which transmits power data to the user device 100 for processing. An interval may be created which requires a user to maintain a specific RPE, and an event has an associated trigger condition which is triggered when the RPE exceeds or falls below a certain predetermined threshold value, or moves out of a predefined range.

Any combination of sensor data or calculated milestone data can be used to create a knowledge function which may be used as a heuristic or ruleset for determining how and whether an event is to be triggered or not. These will be described further below.

### Schedule Playback

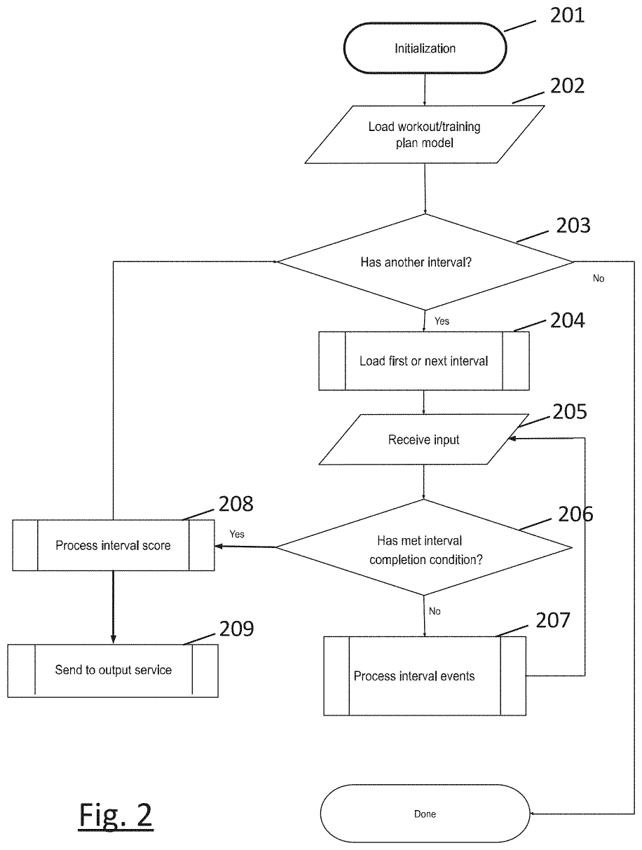

Initialisation and playback of a schedule will now be described, in relation to FIG. 2. The process is initialised at step 201, upon which a workout or training plan model (schedule) is loaded (202) into the feedback schedule engine 120. The schedule engine 120 determines whether the schedule comprises an interval to be completed at 203. If an invalid program is loaded comprising no intervals, then the process ends. The first or next interval is loaded into the engine at 204. The following description assumes that the intervals are processed in series, that is to say only one at a time. The interval comprises a plurality of events, each with one or more associated milestones or event trigger conditions. Sensor data input is received at 205. The feedback schedule engine determines whether the interval completion condition has been met at 206. Each interval has an interval completion condition. The interval completion condition may simply be related to a timer, and in this case will be reached when the allotted time has elapsed. The completion condition may be based on any metric, or knowledge function described herein. Where it is determined that the completion condition has not been met, the feedback schedule engine 120 processes the events which are associated with the interval at 207. When the interval completion condition is met, an interval score is processed at 208. The interval score relates to a defined knowledge function and may be stored in storage, or fed back into the engine to be used in subsequent intervals. The interval performance score may be provided to the user upon completion of the interval. Any generated and received data regarding the interval completion may be stored to be used in a subsequent playback of the schedule, or for use in another schedule to be executed by the user device 100, or other user device, at a later time.

A schedule may also comprise global events which are processed at every stage during the schedule playback. The global events are processed in parallel to interval events and are similarly associated with event trigger conditions.

### Interval Score

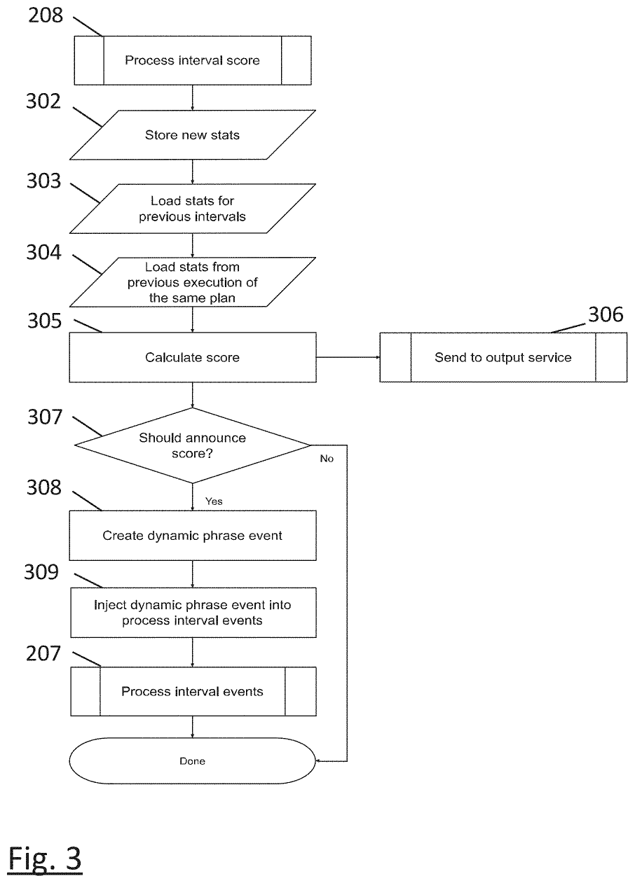

The processing and creation of events based on an interval score will now be described, in relation to FIG. 3. At step 208 the interval processing score begins. The data and statistics relating to the completion of an interval is retrieved from the feedback schedule engine 120 and stored in storage 138. The interval completion data relates to the relevant data for completion of the interval, and this may include any or all of the sensor data and performance data calculated on the basis of the sensor data. For example, for a running based interval, the stored data may include: elapsed time, average pace (/km or /mile), maximum pace, maximum heart rate, average heart rate. At step 303 previous interval data is loaded. Previous interval data relates to interval data for an interval with the same or similar characteristics that has already been completed in the present schedule playback. The interval completion data may refer to an individual interval, or to an interval group, as described above. Where an interval group is completed, one or both of the individual interval data and interval group data may be loaded for comparison. At step 304 historic data from previously completed schedules is loaded, for comparison. The historic data is retrieved for schedules matching the schedule presently being executed, or for schedules comprising intervals that match those of the present schedule. The previous and historic data which is loaded is used to create an interval completion score which relates to the user's relative performance to both the previous intervals performed in the present schedule, and also to historic data relating to intervals from already-completed schedules. Alternatively, historic data may relate general conditions met during previously completed schedules, such as maximum detected heart rate, or fastest pace recorded, for example.

A score is calculated at 305. The score relates to the completion of the interval, and this may be calculated in comparison to the previous or historic data loaded in steps 303 and/or 304. Alternatively, the score may simply relate to instantaneous performance of the completed interval. When the score has been calculated, it is sent to the output service at step 306. At step 307 a determination is made as to whether the calculated score should be announced. The determination may be made based on whether the calculated score differs from the previous or historic interval by meeting or exceeding a predetermined threshold condition. Where it is determined that the score should be announced, a dynamic phrase event is created at step 308. The dynamic phrase event may be created from a repository of pre-recorded phrase portions, stored in feedback element library 132. Once created, the dynamic phrase event is inserted or injected into a subsequent interval in the feedback schedule engine for scheduling and processing as is described further below. Alternatively, the dynamic phrase event may be scheduled as a global event.

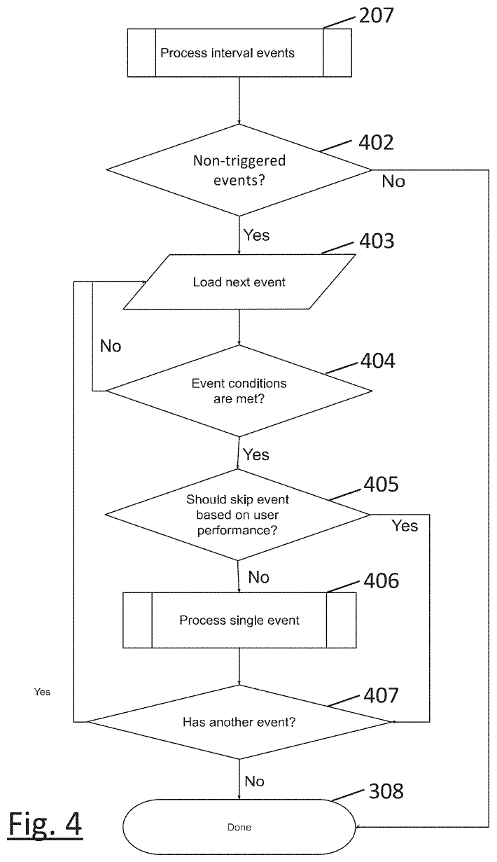

The processing of interval events will now be described in relation to FIG. 4, which shows an example process. At step 207, interval event processing is initialised. A determination as to whether the interval comprises non-triggered events is made at step 402. The next interval event is loaded at step 403. As described in the foregoing, the interval event comprises threshold conditions which must be must for the event to be processed. These are also described as milestones. The determination as to whether the metric threshold conditions are met is made at 404.

At 405 a determination is made as to whether the event and associated event feedback should be skipped. This determination can be made based on a number of factors, as will be described further below. The event may be skipped based on user performance, or based on current music being played, for example. A feedback event may be skipped on the basis that an event of higher priority is required to be played out, or remains in the feedback playback schedule.

Where it is determined that the event should be processed, this is done at step 406, and is described further below. Subsequently, at 407 it is determined whether the interval comprises further events to be processed. If there are further events to be processed, the process returns to step 303. Where no non-triggered interval events exist the process terminates at step 408.

The process outlined in FIG. 4 imagines one event being processed at any given time. It is envisioned that many events may be processed at any one time, each dependent on different threshold conditions, or knowledge functions which are required to be met. For example, within a given interval there may be a pace event which is triggered where a user's pace falls outside a predetermined range. A further pace event might be arranged to be triggered for the opposite condition, i.e. when the user's pace remains within the predetermined range for a certain amount of time. These events may provide the user not only with notification that they are outside the predetermined range, but also with added feedback when they are performing as expected.

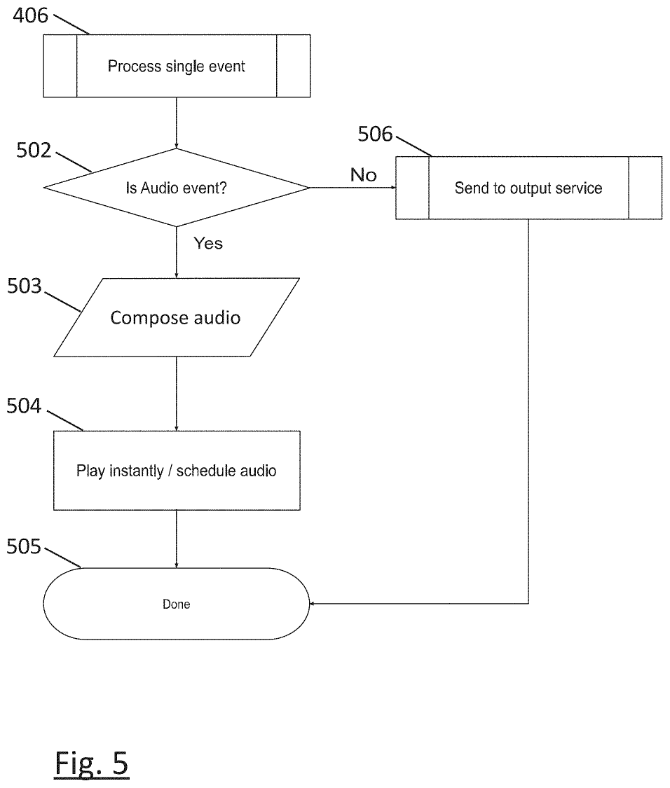

The processing of an event will now be described, with reference to FIG. 5. Event processing begins at step 406. At 502 a determination is made as to whether the event is an audio event or not. If the event is an audio event, obtained or composed at step 503. This step will be described in more detail, below. Once the required audio assets are prepared, at step 504 a determination is made as to whether the assets, or feedback elements, are to be outputted immediately, or whether they are to be scheduled to be played at a later time. If the audio feedback elements are to be played back immediately, they are passed to the audio output generator 142.

Where the event is not an audio event, the feedback element is selected and passed to the output service. The feedback element may be passed to the output service immediately, or maybe scheduled to be presented to the user at a later time. The output module 140 may then provide the feedback element to the user via the non-audio output services, including the display 148, haptic feedback generator 144, transmitter 146.

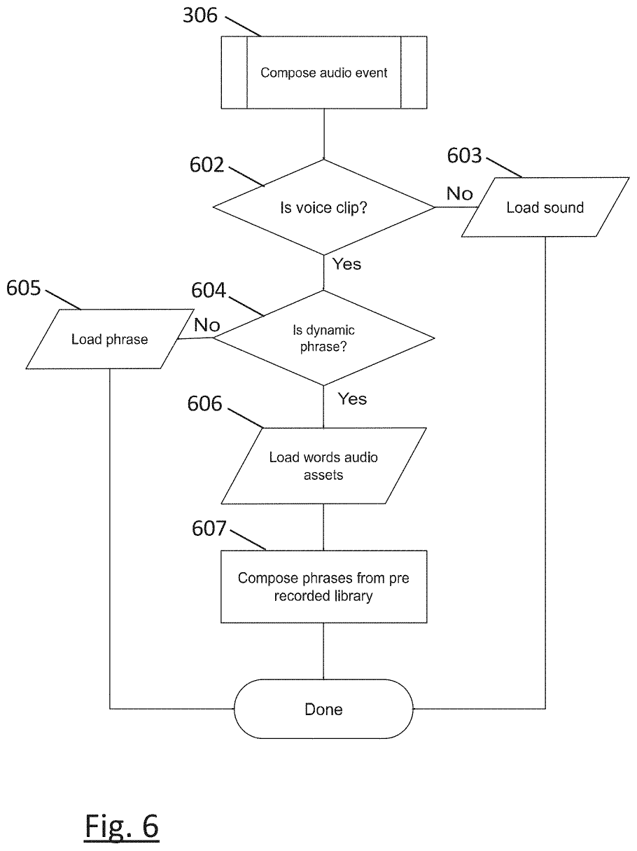

FIG. 6 shows a flow chart describing the process of composing an audio feedback event 503. At step 602 a determination is made as to whether the audio event is a voice clip. Some audio feedback events are associated with voice related feedback, and other audio feedback events may be related with non-speech related audio. In the case that the audio to be played out is not speech-related, the sound is retrieved and loaded in step 603. The process then returns to step 504, as described above in relation to FIG. 5.

If the audio event is a speech-related, or voice element, a determination is then made as to whether the speech-related audio event is a dynamic phrase (604). Many feedback elements relate to items which can be pre-recorded and stored in the memory. If the determination is made that the audio feedback element is not a dynamic phrase—i.e. it relates to a pre-recorded feedback element, the pre-recorded phrase is loaded at step 605 and the process returns to step 504, as described above. If the audio event requires a dynamic phrase, audio assets may be loaded from the feedback element library 132 at step 606. The partial audio assets which have been retrieved from feedback element library 132 are compiled into an appropriate phrase or phrases at step 607. The process then returns to step 504 described above. The audio assets which are retrieved at step 606 may themselves be selected on the basis of one or more knowledge functions, or heuristics, as described above. For example, the audio feedback elements stored in feedback element library 132 may comprise metadata relating to a performance score. The selection of the appropriate audio feedback element may be carried out independent of the most recently calculated interval score. Audio feedback elements which have metadata corresponding to the most recently calculated interval score are selected and one or more phrases are composed from them.

### Knowledge Function/Heuristics

As described above, event trigger functions or milestones may be determined based on any combination of the sensor data received. Examples will now be provided. A simple example is that an event can be triggered when time elapsed is 30 seconds. Another example is where a time elapsed equals a percentage of the total time allotted for an interval. Where an interval is distance based, a knowledge function may be defined as when a predetermined percentage of the total required distance has been completed, as measured by sensor data received from the GPS system 113. A further example uses a combination of altimeter data and barometer data received at the input which are combined to determine that a hill is being climbed at a gradient which is determined to be above a predetermined threshold, and for a time which is longer than a predetermined threshold, and/or at a rate which is higher than a predetermined threshold.

### Event Priority and Scheduling

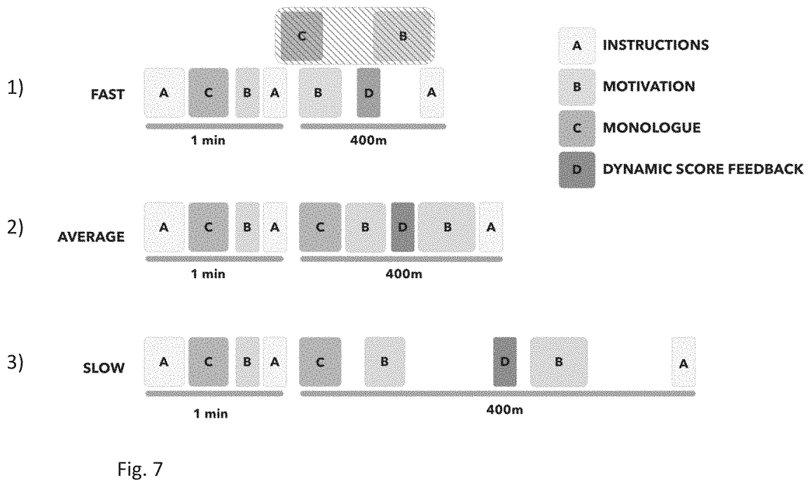

Events may be categorised within a particular interval, or across an entire schedule. One or more events for each interval may be categorised as critical for play out or feedback to the user when an interval is being or is to be completed. Further events may be marked as secondary events, or tertiary events. The prioritisation of events may be prioritised or categorised in a multi-layered approach with any number of priority levels, with relative priority of events being defined according to any logical or algorithmic process accordingly. The categorisation of each event and its relative priority with respect to other events and other classes of events may be provided in event metadata. An interval may therefore comprise one or more critical events, and one or more secondary and or tertiary events. Non-critical events, that is to say secondary or tertiary events may also comprise a user preference weighting. The user preference weighting is also to be considered in the audio playout or scheduling process. An example of the way in which event priority functions is shown in FIG. 7.

In FIG. 7, three simplified scenarios are shown, each comprising a time-based interval (duration:

one minute) and a distance-based interval. Scenario 2 may be considered as the basic schedule, which is played out in normal conditions. Scenarios 1 and 3 relate to cases where a user is performing faster or slower than the basic schedule. The time-based interval across all three scenarios in FIG. 7 comprises 2 critical events, labelled (“A”—instructions), a secondary event (“B”—motivation) and a tertiary event (“C”—monologue). These may all be delivered to the user at the same point of the interval in each scenario, if the trigger conditions for each are met. That is to say, it is predictable when an event is going to happen. In a simple example, all of the trigger conditions may relate to an elapsed time, however the skilled reader will be aware that as we have shown, the actual feedback presented to the user in each scenario may be tailored according to specific sensor data received, or based on previous or stored user data.

The second interval in the basic scenario also comprises a critical event, and further includes a secondary and a tertiary event. The second interval also comprises a dynamic interval (“D”—dynamic score interval). The critical event may have a trigger condition which relates to a distance completed, or distance remaining in the interval. Alternatively, the trigger condition for the critical event may be based on a determination that the user will complete the interval within a specific time period, the determination being based on a knowledge function including any or all of current pace, average pace for the interval distance already completed, historical data relating to a performance score for an interval already completed, or other examples. It can be seen that the dynamic score feedback event is located at the halfway point of the interval, and will be triggered when it is determined that the device has moved the requisite distance to satisfy an event trigger condition. In general, critical feedback and dynamic feedback are locked to a specific position in an interval timeline. The specific position may be located in the timeline as a function of the actual time elapsed, or time remaining in an interval, or a distance that has been covered by the device, a distance remaining in the interval, or a determination of time remaining in the interval, for example. Therefore the critical feedback and dynamic score feedback are triggered in each case when the same amount of interval is elapsed, or remains—that is to say at a fixed point in the progression of the interval.

Based on any combination of user preference data, historic data, interval score, sensor data or event trigger conditions it may be determined that an upcoming interval in a schedule is being, or will be performed at a speed that is faster or slower than the base schedule. As is the case in the “fast” schedule, the distance based schedule which has a length of 400 m will be completed in less time than in the basic, or “average” schedule. In this case the total time required for playback/execution of the planned feedback elements exceeds an estimated time for the completion of the interval, and therefore a determination may be made that the event feedback should not be played out, since it is not possible to complete the playout of the feedback elements during the interval. Alternatively, the determination is made that if a secondary or tertiary event feedback trigger condition is or will be met, the determined performance score, or received and processed sensor data means playout of the secondary or tertiary event feedback will clash with a critical event feedback that is scheduled to be played out at a fixed point in the interval, as described. Since a critical interval must be played out at a fixed point in the interval timeline, and cannot be moved, the secondary or tertiary event feedback output is skipped and not passed to the audio generator 148. Alternatively the clashing secondary or tertiary feedback may be adjusted, by selecting an alternate version from the feedback element library 132, or the feedback may be shortened or truncated so as not to overlap with an immovable or critical feedback element.

To achieve this, the engine continuously monitors performance data based on the received sensor data input, and is arranged to compute a prediction of estimated time remaining for completion of an interval. The prediction of estimated time remaining may be computed at the start of an interval based on previously completed intervals in the schedule currently being executed, or based on historic data relating to intervals having similar performance requirements. This prediction may be updated continuously as the interval progresses based on the received sensor data.

In scenario 1, the “fast” scenario, therefore, then the tertiary events are skipped from the second interval, and only the secondary event and critical events will be passed to the audio generator in the interval. Alternatively a determination may be made that a clashing lower priority event and associated feedback elements are adjustable. For example, a feedback element may be tagged as playable only in part, with a portion of the feedback element tagged as removable if required. This adjusted feedback element may be retained if it is determined that such an adjustment avoids the clash of feedback elements described, and satisfies global system rules regarding separation of feedback elements, and a total amount of a schedule that comprises feedback elements.

In scenario 3, or the “slow” scenario, it is determined that the interval will take longer than in the basic scenario shown in scenario 2. It can be seen that the feedback events of the average scenario are all included, but based on the determined performance score and estimated time remaining, the secondary and tertiary event feedback is delivered to the user at different points in the progression of the interval. The event trigger conditions may be modified at the commencement of the interval based on the performance data or performance score, such that events are not triggered until a later time, or alternatively, when an event is triggered by the trigger condition being met, the scheduler determines an appropriate location to place the audio in an audio playout schedule such that an appropriate spacing of feedback elements is achieved.

The determination that an elapsed interval time is greater than the basic scenario can cause creation or insertion of further events, since the time between feedback elements has grown. For example in scenario 3 shown in FIG. 7, further feedback elements can be provided for between secondary feedback element B and dynamic feedback element D, or likewise between secondary feedback element B and critical feedback element A.

### Scheduling of Feedback

When event milestones or trigger conditions are met, and a feedback element is prepared for output, the feedback element is added to a queue or playout schedule. The feedback queue or feedback schedule is dynamically repurposed and reconfigured as the user progresses through the interval and further event trigger conditions are met. For example, each feedback element has a certain duration. The total duration of the feedback elements that are scheduled to be played out to the user should not exceed the total remaining time in a particular interval. Some feedback elements may be prioritised to play out over an interval boundary, however. For example, when an instruction such as a countdown timer is presented for playout, it will start at the end of an interval, and the “Go” instruction will be presented at the start of the subsequent interval.

When a new feedback element is added to the feedback schedule, similarly to the determination carried out above relating to priority of event feedback, a determination is made as to whether the duration of feedback elements to be played out exceeds a predetermined threshold. In the case that the threshold is exceeded, the feedback schedule may be reconfigured. The reconfiguration may comprise determining a priority level of the newly inserted feedback element and determining whether any feedback elements of a lower priority are present in the queue. If feedback elements of a lower priority are present, one or more of the lower priority elements may be skipped from the feedback schedule.

As will be appreciated, although an interval may be commenced with a number of events that are to be processed based on the basic schedule, depending on the user's performance, the trigger conditions will be met at different times throughout the interval. Feedback elements that are to be associated with the events from an interval are not only delivered at different times during execution of the schedule, but the selection of the feedback to be played out is dependent on both the user's actual performance of the interval, and based on their performance of the interval relative to stored data, in relation to estimated interval completion metrics calculated by the feedback schedule engine 120.

The determination as to whether to skip a feedback element may be made based on user preference data that may be stored in storage 130, or input by a user at the initiation of a schedule. The user preference data may relate to a weighting to be applied to secondary and tertiary feedback elements. Users may wish for more or fewer secondary or tertiary feedback elements to be passed to the output for playback during execution of a schedule. The weighting may be applied to any non-critical interval, and if a clash is determined to exist between any two feedback elements that may be scheduled, a combination of the weighting factor applied, as well as the feedback element priority level is taken into account when determining to skip the playout of a feedback element. Furthermore, a user may select that only a specific type of feedback should be presented, or skipped—for example a user may wish to only allow haptic feedback events to be presented. Once a feedback element is skipped, it cannot be re-added to the audio queue

The prioritising of feedback elements and scheduling of feedback elements has been described in relation to a particular interval such as that shown in FIG. 7. A schedule may also comprise global events with their own associated trigger conditions which are processed by the feedback schedule engine at all times during the playout of the schedule. By their nature, the global events will not be associated with fixed points in the schedule, either based on time or amount of an interval completed. Nonetheless, a global event may be prioritised as a critical event which causes the modification of the schedule when the trigger conditions of the global event are met. A global event can be prioritised at any level. Where a global event trigger condition is satisfied, the feedback schedule engine determines, based on the events already present in the interval and their associated feedback elements, as well as the predicted interval performance score and estimated remaining time, whether and where to insert the global event feedback element into the feedback playout schedule. Insertion of the global event feedback may cause the skipping of event feedback for an event associated with the current interval. It may be determined that the global event feedback should be placed in an interval that is scheduled, rather than the present interval.

As has been described, when an event trigger condition is met, it may give rise to the creation of further events, to be placed at a later point in the schedule. For example, an interval group may comprise 6 distance based intervals, each of the same distance. The third interval comprises an event whose trigger condition is triggered when the user completes the third interval. When triggered, the metric threshold conditions are reviewed and it is determined that the first three intervals were performed within a predetermined metric range. This determination may be based on user preference data, or may be based on a simple threshold defined in interval metric data, or from a calculation with respect to historic interval data stored in storage 130. The metric threshold condition is met, and an event is created in the fourth interval, with associated new trigger condition and associated feedback elements. The event is prioritised as a secondary event, and the feedback schedule engine determines that an event already located in the fourth interval has a lower priority, and therefore marks that event as to be skipped. Furthermore, the trigger condition of the newly created interval is determined according to rules regarding the amount of concurrent feedback to be provided to a user (minimum silence or space between feedback elements, for example) and this causes the adjustment of the trigger condition of a further event in the fourth interval, such that it is triggered at a later time in the interval, thereby avoiding a schedule clash.

Embodiments of the invention described herein relate to a system and method for the dynamic modification of a feedback schedule, based on sensor data relating to an electronic user device. The invention creates an audio output schedule that can be modified, by addition, deletion or re-ordering of elements in the queue based on directly received sensor data, metrics determined from the received sensor data and other predefined characteristics with which feedback elements are associated, as described herein. In some embodiments, the system and method provide for the dynamic creation and playout of an audio file that advantageously avoids clashing of audio feedback elements that are to be played out by an audio output. Both pre-recorded elements and dynamically created audio elements are created in a playout schedule based on a combination of sensor data, a hierarchy of feedback elements and intelligent scheduling, as described above.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “ receiving”, “determining”, “comparing”, “enabling”, “maintaining”, “identifying”, “selecting”, “allocating” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A computer-implemented method for controlling playback of a schedule of feedback elements to be presented at an output of an electronic device, the feedback elements each associated with one or more predefined metrics, the method comprising, during playback of the schedule:
receiving sensor data from one or more sensors associated with the electronic device;
upon determining that a first trigger condition is satisfied, determining whether the sensor data satisfies a metric threshold condition,
in response to the metric threshold condition being satisfied, modifying the schedule of feedback elements for presenting at the output.

2. The method of claim 1, wherein the schedule comprises a first feedback element associated with a first metric, and modifying the schedule comprises providing a second feedback element associated with the first metric, and replacing the first feedback element with the second feedback element.

3. The method of claim 2, wherein the first feedback element is associated with a first range of first metric data and the second feedback element is associated with a second range of first metric data, and selecting the second feedback element is done when the satisfied metric threshold condition is located in the second range.

4. The method of claim 1, wherein the schedule comprises first and second feedback elements and wherein the step of modifying the schedule further comprises adding a third feedback element to the schedule for presenting at the output.

5. The method of claim 1, wherein modifying the schedule comprises modifying a time at which a scheduled feedback element is to be presented at the output.

6. The method of claim 1, wherein the schedule comprises a schedule of events, each having an associated trigger condition and one or more associated feedback elements, and modifying the schedule comprises, when a first associated trigger condition of a first event is satisfied, removing the associated feedback element of a second event from the schedule of feedback elements for presenting at the output.

7. The method of claim 1, wherein the schedule further comprises,
a schedule of events, each comprising an associated trigger condition and associated with one or more of the predefined metrics, wherein modifying the schedule comprises, where a first associated trigger condition of a first event is satisfied, creating a second event comprising a second associated trigger condition and associated with one or more of the predefined metrics, and
adding the second event to the schedule of events.

8. The method of claim 1, wherein the schedule of feedback elements comprises at least one predetermined feedback element, and modifying the schedule comprises:
generating a context-dependent feedback element based on the sensor data, and
adding the generated context-dependent feedback element to the schedule of feedback elements for presenting at the output.

9. The method of claim 8, wherein generating the context-dependent feedback element comprises combining a plurality of media samples.

10. The method of claim 1, wherein determining whether the metric threshold condition is satisfied comprises comparing the sensor data with stored data associated with one or more of the predefined metrics.

11. The method of claim 1, wherein the feedback elements comprise audio components.

12. The method of claim 1, wherein the feedback elements comprise speech components.

13. The method of claim 1, wherein the schedule of feedback elements comprises a plurality of intervals each having a length, and a first and second interval boundary, and wherein the first trigger condition is satisfied when during playback of the schedule an interval boundary is crossed.

14. The method of claim 13, wherein the length of an interval is defined as one of: a function of time or a function of distance.

15. The method of claim 1, wherein the sensor data relates to at least one of:
biometric data gathered by a biometric sensor;
location data gathered by a location sensing device; or
movement data gathered by an inertial measurement unit.

16. The method of claim 1, wherein the predefined metrics comprise one or more user parameters, the user parameters being at least one of:
pace;
speed;
heart rate;
cadence;
stroke rate;
volume of oxygen consumption (VO2); or interval completion success.

17. A computer readable medium comprising computer readable instructions configured, in use, to enable a processor to perform the method of claim 1.

18. An electronic device arranged to playback a schedule of feedback elements, the electronic device comprising a processor arranged to perform the method of claim 1.

19. The electronic device of claim 18, comprising an audio output, wherein the feedback elements comprise audio data and the output comprises an audio output and wherein playback of the schedule of feedback elements comprises presenting the feedback elements at the audio output.
